(19) United States
(12) Patent Application Publication
(10) Pub. No.: US 2024/0302383 A1
(43) Pub. Date: Sep. 12, 2024

(54) PEPTIDE BIOMARKER FOR IDENTIFYING HIPPOCAMPUS SPECIES BASED ON PEPTIDOMICS AND APPLICATION THEREOF

(71) Applicant: SHANDONG INSTITUTE FOR FOOD AND DRUG CONTROL, Jinan (CN)

(72) Inventors: Yongqiang LIN, Jinan (CN); Yang JIAO, Jinan (CN); Li SHI, Jinan (CN); Xue YIN, Jinan (NG); Yingying XIE, Jinan (CN); Bing WANG, Jinan (CN); Fei XUE, Jinan (CN); Lili XU, Jinan (CN)

(73) Assignee: SHANDONG INSTITUTE FOR FOOD AND DRUG CONTROL, Jinan (CN)

(21) Appl. No.: 18/596,683

(22) Filed: Mar. 6, 2024

(30) Foreign Application Priority Data
Mar. 10, 2023 (CN) 202310244892.9

(51) Int. Cl. G01N 33/68; C12Q 1/37; G01N 1/28

(52) U.S. Cl. CPC G01N 33/6848; C12Q 1/37; G01N 1/286; G01N 2333/4603

(57) ABSTRACT

A peptide biomarker for identifying Hippocampus species based on peptidomics and application thereof are disclosed, belonging to the technical field of biology. The protein in Hippocampus is studied, and the Hippocampus thermostable protein is comprehensively analyzed using a peptidomics method. The analysis discovered potential peptide biomarkers of Hippocampus collagen, the specificity of the potential peptide biomarkers is verified by HPLC-triple quadrupole mass spectrometry, and the peptide biomarkers are comprehensively identified by combining bioinformatics analysis. Finally, 10 peptide biomarkers are discovered, targeting 11 different species of Hippocampus. The markers provided may identify Hippocampus existing in the market, promote the scientific and standardized classification and grading of Hippocampus commodities, and play an important role in high-quality and high-price trade circulation of the Hippocampus commodities.

### CROSS-REFERENCE TO RELATED APPLICATIONS

This application claims priority to Chinese Patent Application No. 202310244892.9 with a filing date of Mar. 10, 2023. The content of the aforementioned application, including any intervening amendments thereto, is incorporated herein by reference.

### CROSS-REFERENCE TO A SEQUENCE LISTING

This application includes a Sequence Listing submitted electronically as a text file named SEQ-list.xml, created on Mar. 1, 2024, with a size of 9,915 bytes. The Sequence Listing is incorporated by reference herein.

### FIELD OF THE INVENTION

The present invention belongs to the technical field of biology, and particularly relates to a peptide biomarker for identifying Hippocampus species based on peptidomics and application thereof.

### BACKGROUND OF THE INVENTION

Hippocampus exhibits special morphological and life history characteristics, such as a graspable tail, a slender nose, headgear without scales, absence of caudal and ventral fins, and a unique pattern of male pregnancy. When reaching a new environment, Hippocampus exhibits physical characteristics that quickly adapt to the new environment, including body size, color patterns, etc. For example, the presence or absence of spines may be an adaptation to predators, which involves the evolution of new species. Hippocampus is currently known as the species with the fastest evolution rate; therefore, it has a large family of species, with few species differences. It has important medicinal values and is a traditional Chinese medicinal material for strengthening and tonifying.

According to statistics, Hippocampus is traded globally in large quantities, with trade in dried Hippocampus accounting for 98% of the total trade, and dried Hippocampus is primarily used in the trade of traditional medicines. Since the 1980s, the yield of Hippocampus has been declining due to the pollution of ecological environment and the restriction of natural resources.

Currently, methods for identifying Hippocampus species mainly rely on the appearance identification and molecular biology identification. During the circulation process, dried Hippocampus is the primary form of trade. However, during the drying or transportation process, Hippocampus loses many appearance characteristics for species identification due to abrasion and bumping, such as speckles, protrusions, and links, making it difficult to identify the species through observation. The molecular biology identification method has higher specificity than the traditional appearance identification method, but it also has limitations. Particularly when Hippocampus is mixed and crushed with or cooked by heating, the molecular biology identification method cannot distinguish its species. Hippocampus has a wide range of medicinal values and is also the raw material of Hippocampus glue. However, due to the similarity of Hippocampus appearance and physicochemical properties, there are many problems in the traditional identification of Hippocampus species.

### SUMMARY OF THE INVENTION

The present invention provides a peptide biomarker for identifying Hippocampus species based on peptidomics in view of the blank existing in the related art for identifying Hippocampus species using a peptide marker.

The present invention also provides application of the above-mentioned peptide biomarker in identifying Hippocampus species.

The technical solution adopted by the present invention to achieve the above-mentioned object is as follows.

The present invention provides a peptide biomarker for identifying Hippocampus species based on peptidomics, and a sequence of the peptide biomarker is as follows:

| Peptide fragment | Sequence |
|---|---|
| Pep1 (SEQ ID NO. 1) | VGPAGPAGAGGPP(+15.99)GPGGPVGK |
| Pep2 (SEQ ID NO. 2) | GPP(+15.99)GAQGAAGAP(+15.99)GPK |
| Pep3 (SEQ ID NO. 3) | GPTGEIGATGPVGSR |
| Pep4 (SEQ ID NO. 4) | SGEQGVP(+15.99)GEAGAP(+15.99)GVAGSR |
| Pep5 (SEQ ID NO. 5) | GELGHTGPDGNAGR |
| Pep6 (SEQ ID NO. 6) | GEAGAP(+15.99)GEDGIP(+15.99)GSMGAR |
| Pep7 (SEQ ID NO. 7) | GETGAAGENGTP(+15.99)GAMGPR |
| Pep8 (SEQ ID NO. 8) | VGPAGPAGVAGPP(+15.99)GPAGPVGK |
| Pep9 (SEQ ID NO. 9) | GEAGAAGENGLP(+15.99)GAMGPR |
| Pep10 (SEQ ID NO. 10) | GETGPAGPVGSAGAR |

Further, the peptide biomarker is configured to distinguish Hippocampus kuda, Hippocampus kelloggi, Hippocampus trimaculatus, Hippocampus histrix, Hippocampus mohnikei, Hippocampus spinosissimus, Hippocampus comes, Hippocampus ingens, Hippocampus fuscus, Hippocampus camelopardalis, and Hippocampus reidi.

The present invention also provides application of the above-mentioned peptide biomarker in identifying Hippocampus specie, and identification principles are as follows:
(1) when only Pep1 is detected in a sample, the sample is identified as Hippocampus histrix;
(2) when only Pep2 is detected in a sample, the sample is identified as Hippocampus kelloggi;
(3) when only Pep3 is detected in a sample, the sample is identified as Hippocampus trimaculatus;
(4) when only Pep4 is detected in a sample, the sample is identified as Hippocampus mohnikei;
(5) when only Pep5 is detected in a sample, the sample is identified as Hippocampus kuda and Hippocampus reidi;
(6) when only Pep6 is detected in a sample, the sample is identified as Hippocampus spinosissimus;
(7) when only Pep7 is detected in a sample, the sample is identified as Hippocampus comes;
(8) when only Pep8 is detected in a sample, the sample is identified as Hippocampus ingens;
(9) when only Pep9 is detected in a sample, the sample is identified as Hippocampus fuscus;
(10) when only Pep10 is detected in a sample, the sample is identified as Hippocampus camelopardalis.

Preferably, a specific identification process is as follows:
(1) Sample preparation
weighing a Hippocampus extract accurately, adding water, sonicating for dissolving, cooling to room temperature, measuring a supernatant accurately, and adding trypsin to obtain a test solution after enzymolysis overnight;
(2) using High Performance Liquid Chromatography (HPLC)-triple quadrupole mass spectrometry for analysis

Further, in step (1), a specific preparation process of the Hippocampus extract is as follows: taking an appropriate amount of test Hippocampus and placing in a triangular flask, adding water to soak for 48 h, and changing the water once in the middle; adding 250 ml of water to desalted Hippocampus and decocting at high temperature 3 times for 4 h, 3 h, and 2 h; combining decocted solutions, concentrating decocted solutions by slight boiling until they are viscous, transferring to a silica gel bowl, and drying to solid in a 60° C. electrothermal constant temperature air blast drying oven.

Further, in step (1), a ratio of the Hippocampus extract to water is 0.01 g: 5 mL; a time of the sonicating is 30 min; a volume ratio of the supernatant to trypsin is 50:1; a concentration of trypsin is 1 mg/ml; a temperature of the enzymolysis is 37° C.

Further, in step (2), parameters of the HPLC-triple quadrupole mass spectrometry are as follows: chromatographic column Agilent Eclipse C18 (2.1×100 mm, 1.8 μm), a mobile phase including A (0.1% formic acid aqueous solution) and B (0.1% formic acid acetonitrile solution) for gradient elution; injecting 5 μl of samples at a flow rate of 0.3 ml/min.

Further, the gradient elution is specifically as follows: 0-20 min, 3%-20% B; 20-21 min, 20%-90% B; 21-24 min, 90%-3% B; 24-30 min, 3% B.

Further, the parameters of the triple quadrupole mass spectrometry are as follows: a mode being set as a mass detector, with electrospray ionization (ESI) and positive ion multiple reaction monitoring; a flow rate of sheath gas being 46 L/h, a flow rate of auxiliary gas being 850 L/h, a spray voltage being 3.5 kV, a source temperature being 150° C., a temperature of auxiliary gas being 400° C., a cone voltage being 30 V, a collision voltage being 35 V, and a solvent delaying 0-1 min and 21-30 min for use.

In the present invention, the protein in Hippocampus is studied, and the Hippocampus thermostable protein is comprehensively analyzed using a peptidomics method. The analysis discovered potential peptide biomarkers of Hippocampus collagen, the specificity of the potential peptide biomarkers is verified by HPLC-triple quadrupole mass spectrometry, and the peptide biomarkers are comprehensively identified by combining bioinformatics analysis. Finally, 10 peptide biomarkers are discovered to distinguish Hippocampus kuda, Hippocampus kelloggi, Hippocampus trimaculatus, Hippocampus histrix, Hippocampus mohnikei, Hippocampus spinosissimus, Hippocampus comes, Hippocampus ingens, Hippocampus fuscus, Hippocampus camelopardalis, and Hippocampus reidi.

Beneficial effects of the present invention are as follows.

(1) The present invention uses a non-targeted mass spectrometry method to analyze Hippocampus collagen peptides, combined with chemometrics and datasets to discover potential peptide biomarkers. HPLC-Triple Quadrupole is used to verify the specificity and establish the identification method. A total of 10 peptide biomarkers have been discovered, targeting 11 different species of Hippocampus. (2) Currently, the database of Hippocampus species only has Hippocampus comes, and there is no independent database of other Hippocampus, which has considerable challenges for the identification of potential peptide biomarkers. This established strategy may identify Hippocampus existing in the market and play an important role in regulating the circulation of Hippocampus samples in the market.

### BRIEF DESCRIPTION OF THE DRAWINGS

FIG. 1 is a diagram of appearances and shapes of different Hippocampus;

FIG. 2 is a strategy chart of an analysis solution for identifying potential biomarkers of Hippocampus;

FIG. 3 shows the sequence alignment of 10 peptide biomarkers selected by the present invention;

FIG. 4 is a mass spectrum of 10 peptide biomarkers selected by the present invention;

FIG. 5 is a mass spectrometry picture of a mixed control solution; and

FIG. 6 is a typical map of Hippocampus species screening in Bushenning tablets.

### DETAILED DESCRIPTION OF THE EMBODIMENTS

The technical solution of the present invention is further explained and illustrated below by specific embodiments.

### (I) Materials and Methods

### (1) Reagents and Materials

Trypsin (sequencing grade) is purchased from Sigma-Aldrich (St. Louis, MO, USA), formic acid (optima LCMS) is purchased from Thermo Fisher Scientific (Waltham, MA, USA), acetonitrile is purchased from Merck KGaA (Darmstadt, Germany), and water is prepared on a MilliQ A 10 Gradient system from Millipore (Schwalbach, Germany).

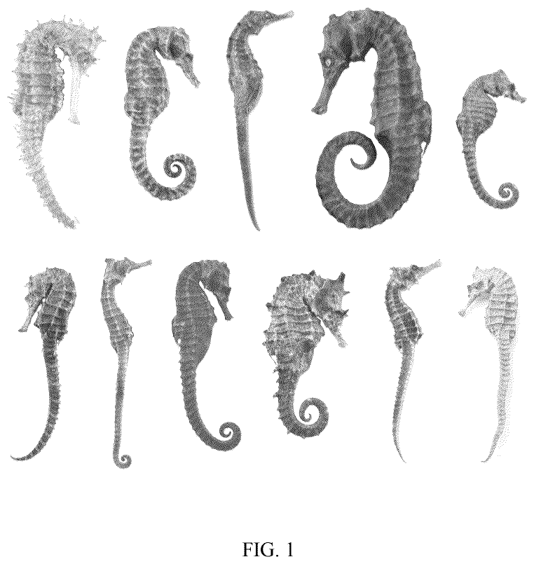

Eleven batches of Hippocampus samples are collected from markets in different regions of China. The appearances and shapes of different Hippocampus are shown in FIG. 1 (the first row from left to right is Hippocampus histrix, Hippocampus kuda, Hippocampus trimaculatus, Hippocampus kelloggi, and Hippocampus mohnikei; the second row from left to right is Hippocampus comes, Hippocampus ingens, Hippocampus fuscus, Hippocampus camelopardalis, Hippocampus spinosissimus, Hippocampus reidi). Detailed information of the samples is shown in table 1. Eleven Hippocampus are used to discover peptide biomarkers, and all samples are used for validation. The peptide biomarkers shown in table 1 are synthesized by ChinaPeptides (Shanghai, China) and GL Biochem (Shanghai, China).

TABLE 1
Detailed information of 10 peptide biomarkers

| | | | | m/z | m/z | |
|---|---|---|---|---|---|---|
| Species | Number | Peptides | Mass | Primary | Secondary | Z |
| H.histrix | Pep1 | VGPAGPAGAGGPP(+15.99)GPGGPVGK | 1670.1 | 835.4 | 992.5 | 2 |
| H.kelloggi | Pep2 | GPP(+15.99)GAQGAAGAP(+15.99)GPK | 1263.6 | 633.3 | 244.1 | 2 |
| H.trimaculatus | Pep3 | GPTGEIGATGPVGSR | 1355.4 | 678.5 | 572.3 | 2 |
| H.mohnikei | Pep4 | SGEQGVP(+15.99)GEAGAP(+15.99) | 1714.7 | 857.9 | 1157.6 | 2 |
| | | GVAGSR | | | | |
| H.kuda, H.reidi, | Pep5 | GELGHTGPDGNAGR | 1337.3 | 446.5 | 474.2 | 3 |
| H.spinosissimus | Pep6 | GEAGAP(+15.99)GEDGIP(+15.99) | 1660.7 | 830.8 | 638.3 | 2 |
| | | GSMGAR | | | | |
| H.comes | Pep7 | GETGAAGENGTP(+15.99)GAMGPR | 1645.7 | 823.8 | 974.4 | 2 |
| H.ingens | Pep8 | VGPAGPAGVAGPP(+15.99)GPAGPVGK | 1725.9 | 863.9 | 706.5 | 2 |
| H.fuscus | Pep9 | GEAGAAGENGLP(+15.99)GAMGPR | 1627.7 | 814.4 | 985.5 | 2 |
| H.camelopardalis | Pep10 | GETGPAGPVGSAGAR | 1283.3 | 642.5 | 939.5 | 2 |

### Example 1

### (1) Sample Preparation

An appropriate amount of test Hippocampus was taken and placed in a triangular flask, water was added to soak for 48 h, and the water was changed once in the middle. 250 ml of water was added to desalted Hippocampus, decocting at high temperature 3 times for 4 h, 3 h, and 2 h. Decocted solutions were combined and concentrated by slight boiling until they are viscous, transferred to a silica gel bowl, and dried to solid in a 60° C. electrothermal constant temperature air blast drying oven. The solid was taken as a Hippocampus extract. 0.01 g of Hippocampus extract was weighed accurately, 5 ml of water was added, sonicated for 30 min for dissolving, and cooled to room temperature. 500 μl of supernatant was measured accurately, and 50 μl of trypsin (1 mg/ml) was added to obtain a test solution after enzymolysis overnight at 37° C.

### (2) Nano-LC-MS/MS Analysis

Prepared Hippocampus samples were analyzed using nano-liter liquid phase (EASY-nLC 1000, Thermo Scientific, San Jose, CA, USA) coupled with high resolution mass spectrometry (Orbitrap-Fusion, Thermo Scientific, San Jose, CA, USA). Desalting enrichment was carried out on a Thermo Acclaim PepMap C18 column (100 μm×3.5 cm, 5 μm, Thermo Scientific, San Jose, CA, USA), and separation on a Thermo Acclaim PepMap C18 (75 μm×15 cm, 3 μm, Thermo Scientific, San Jose, CA, USA). 2 μl of samples were injected at a flow rate of 300 nl/min, and gradient elution was carried out with water (A) with a formic acid concentration of 0.1% (v/v) and acetonitrile (B) with a formic acid concentration of 0.1% (v/v): 0-1 min, 99%-94% A(v/v); 1-96 min, 94-78% A(v/v); 96-113 min, 78-70% A(v/v); 113-117 min, 70-5% A(v/v); 117-120 min, 5% A. Analysis was carried out using a Fusion-Orbitrap mass spectrometry system equipped with a Nanospray Flex in positive ion mode with a spray voltage of 2.1 kV, an ion transport capillary temperature of 275° C., and an S-Lens transport efficiency set to 60%. A primary mass spectrometry used an Orbitrap as a mass analyzer with a resolution of 60,000 (m/z=400) and an acquisition range of 350-1,550 (m/z). A secondary mass spectrometry used an Orbitrap as a mass analyzer and was scanned in Data-Dependent MSn Scan mode and fragmented in HCD mode with a fragmentation energy HCE set to 30%.

### (3) Mass Spectrometry Data Analysis and Selection of Species Peptide Biomarkers

Data in mass spectrometry Raw format was peak aligned and framed using SIEVE 2.2, and a search mode was selected as Defer identification. Identification results were shown as a peak area of the unique component corresponding to each m/z and RT in each species, as well as a ratio, a variance, and a P value of the peak area of each sample to the control sample. The identification results were exported for chemometric analysis including principal component analysis (PCA) and orthogonal partial least squares discriminant analysis (OPLS-DA) using Simca 14.1 (32-bit) software. The PCA is an unsupervised data dimensionality reduction method, and the OPLA-DA is an extension of partial least squares discriminant analysis (PLS-DA). The PLS-DA can maximize the differences between groups according to pre-defined classification and obtain a better separation effect than PCA. The OPLS-DA can filter the changes unrelated to experimental conditions. Therefore, the OPLS-DA can reflect the sample differences related to experimental conditions more than the PLS-DA so that the sample separation effect between groups is better. In an OPLS-DA model, potential characteristic ions of each species were found by pairwise alignment.

Peaks studio8.5 identification results were exported, and datasets suspected of having the same components were selected. All the components identified by mass spectrometry data in the 11 samples were recognized as dataset A. The filtering criteria were as follows: the m/z between any two components was the same (specifically to two decimal places), and the retention time difference was less than 5 min. The data satisfying these two conditions were summarized in dataset B. Dataset C, the complement of dataset A relative to dataset B, was the potential peptide biomarker we were looking for. Potential peptide biomarkers of each species were ranked by response size, and the specificity was verified sequentially.

### (4) Verification of Specificity of Peptide Biomarkers by HPLC-MS/MS

All samples were validated using an AB sciex triple quadrupole mass spectrometer with multiple reaction monitoring (MRM) analysis connected to an ESI source. A chromatographic column Agilent Eclipse C18 (2.1×100 mm, 1.8 μm) was used to carry out chromatographic separation on the sample, and a mobile phase includes A (0.1% formic acid aqueous solution) and B (0.1% formic acid acetonitrile solution), where 0-20 min, 3%-20% B; 20-21 min, 20%-90% B; 21-24 min, 90%-3% B; 24-30 min, 3% B. 5 μl of samples was injected at a flow rate of 0.3 ml/min. The chromatographic column was kept at a constant temperature of 43° C. and equilibrated at initial conditions (3% B) for 2 min.

The LC-MS/MS system was controlled using the Analyst Software. The parameters were set as follows: a mode was set as a mass detector, with electrospray ionization (ESI) and positive ion multiple reaction monitoring; a flow rate of sheath gas was 46 L/h, a flow rate of auxiliary gas was 850 L/h, a spray voltage was 3.5 kV, a source temperature was 150° C., a temperature of auxiliary gas was 400° C., a cone voltage was 30 V, a collision voltage was 35 V, and a solvent delayed 0-1 min and 21-30 min.

### (5) Comprehensive Identification of Specific Peptide Biomarkers

Resulting MS/MS data were analyzed using Peaks Studio software (8.5 Edition, Bioinformatics Solutions Inc., Waterloo, Canada), and searched using the database of Hippocampus comes collagen downloaded from NCBI (downloaded Dec. 21, 2021). Trypsin was selected to allow up to 6 missing cuts. Oxidation (+15.99), hydroxylation (+15.99), desamidization (+0.98), acetylation of the N-terminus of the protein (+42.01), carbamylation (+57.02), and acetylation of lysine (+42.01) were designated as variable modifications. All other parameters are default, including a maximum parent ion tolerance of 10 ppm and a fragment ion tolerance of 0.02 Da.

Results with ALC score greater than 95% in results of PEAKS spider were selected, theoretical fragment ions and actual fragment ions were aligned and further confirmed using a mass spectrometry MRM mode and a Product Ion (MS2) mode. Meanwhile, a basic local alignment search tool (BLAST) search was carried out to assist in verifying the accuracy of the selected peptide fragments.

### (6) Synthesis and Verification of Peptides

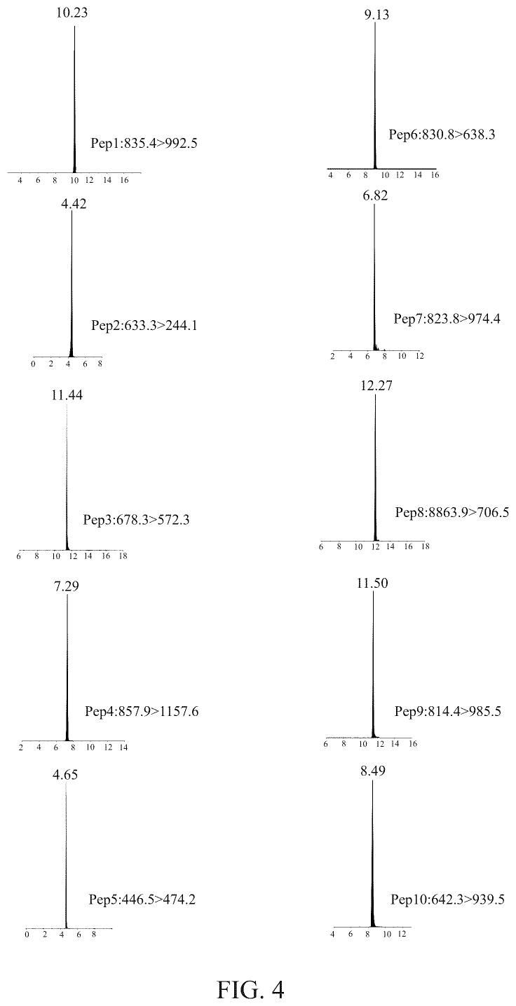

The synthesized peptide biomarker was made into 1 ug/ml solution with ultrapure water. To verify the accuracy of a sequence of the synthetic peptide, a HPLC-triple quadrupole MS was used for the analysis of the synthetic peptide and the Hippocampus preparation sample in an injection volume of 5 μL. An AB sciex mass spectrometer connected to the ESI source was used, and the samples were separated using the Agilent Eclipse C18 chromatographic column (2.1×100 mm, 1.8 μm). The method of step (4) was used to sequentially inject the Hippocampus preparation sample, the synthetic peptide, and the marked sample. If a peak time of the synthetic peptide matches a peak time of the Hippocampus sample, and the synthetic peptide and the Hippocampus sample have only one peak, the sequence of the peptide biomarker may be identified to match the sequence of the Hippocampus sample. A characteristic polypeptide mass spectrum provided by the present invention is shown in FIG. 4.

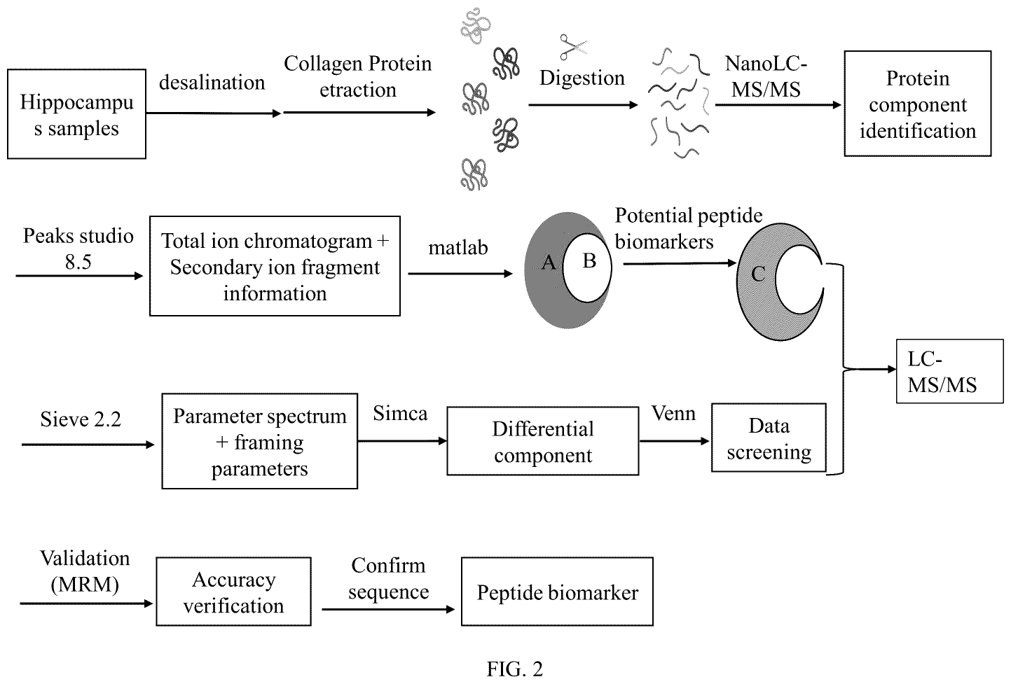

(I) Verification of effects
(1) FIG. 2 shows a strategy for identifying the peptide biomarkers proposed by the present invention, mainly including the following four steps. (a) A peptide profile was obtained from each sample to fully identify the peptides. (b) Pre-processing of mass spectrometry data contained in peptide profiles was carried out using metabolomics software, and polypeptide information contained in peptide profiles was parsed using database searches. (c) Combined with the chemometric method, the potential peptide biomarkers were discovered by pairwise comparison and dataset analysis. (d) Combined with bioinformatics analysis, potential specific peptide biomarkers were comprehensively identified.
(2) Species-specific peptide biomarkers and application thereof

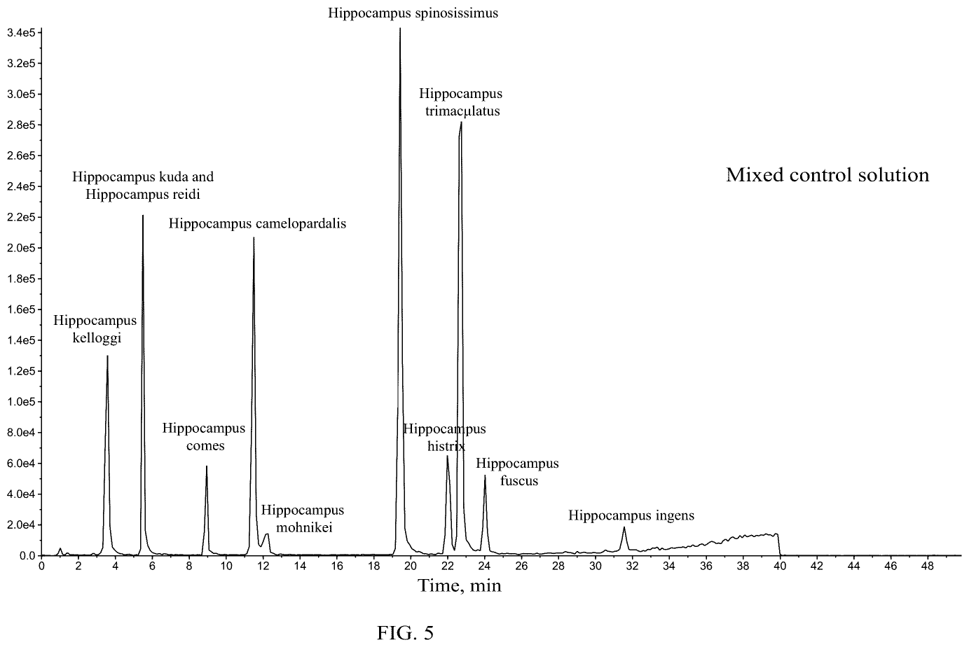

Finally, 10 potential peptide biomarkers were discovered, and detailed information is shown in table 1. Within the 11 species identified in the present invention, pep1-10 are unique components in each species and may be used as identity markers for these species. Two peptide biomarkers for Hippocampus reidi and Hippocampus kuda share the same sequence, possibly due to the high similarity of collagen in the two species. A mass spectrometry picture of a mixed control solution added and a mass spectrometry picture of the individual Hippocampus species are shown in FIG. 5.

### Effect Example

### (I) Identification of Hippocampus as Medicinal Material in Bushenning Tablets

4 g of Bushenning tablet powder was weighed accurately, and 40 mL of water was added accurately, mixed well, and heated to reflux for 1 h. The mixture was taken out and cooled, transferred to a centrifuge tube, centrifuged for 15 min at 12000 r/min, and then transferred to a new centrifuge tube. A subsequent filtrate was obtained by filtering with a 0.22 μm microporous membrane. A certain amount of subsequent filtrate was taken, and a trypsin solution (1 ug/ml) was added in a ratio of 10:1. Enzymolysis was carried out overnight for 12 hours, followed by boiling for 10 minutes to inactivate the trypsin, obtaining a test sample. The test sample was identified using the identification method provided in example 1.

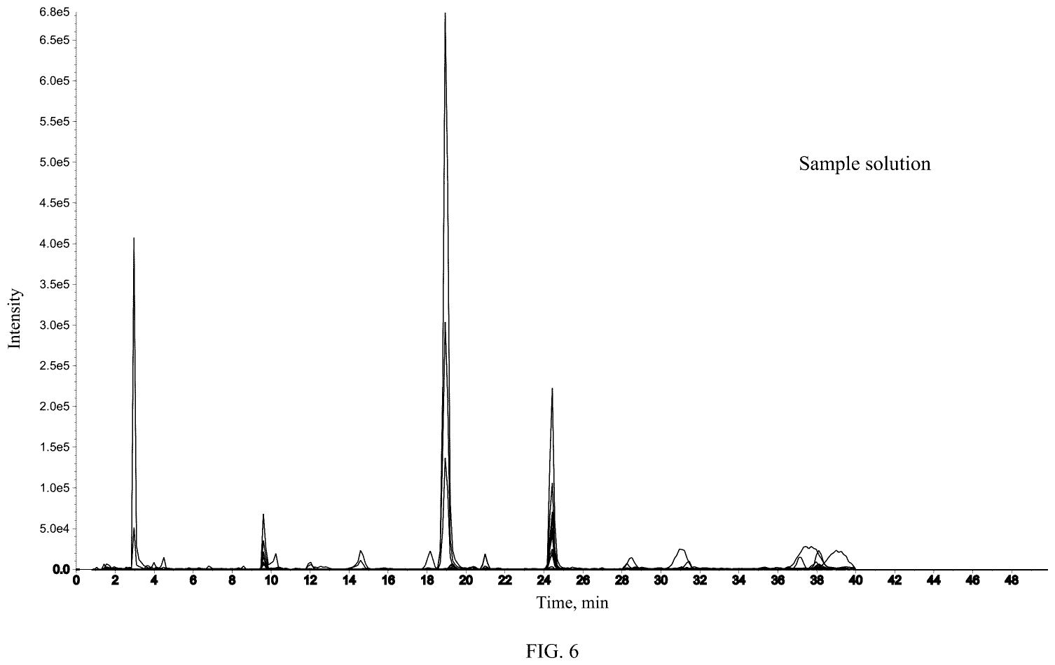

Through the identification of Hippocampus species in Bushenning tablets, it was found that only one batch of samples contained single species information, while two or more species information was found in the other batches of samples (FIG. 6 and table 2). A total of 5 Hippocampus species were found in the samples, i.e., Hippocampus kelloggi, Hippocampus trimaculatus, Hippocampus mohnikei, Hippocampus comes, and Hippocampus spinosissimus. Hippocampus comes and Hippocampus spinosissimus were non-pharmacopoeia species. The experimental results showed that species mixing of Hippocampus was very common, with Hippocampus comes and Hippocampus spinosissimus as the most common species mixed due to the dominance of the number of species.

TABLE 2
Hippocampus species detection results
in Bushenning tablet samples

| | Manufactur- | | |
|---|---|---|---|
| Num- | ing | Batch | |
| ber | enterprise | number | Hippocampus species |
| 1 | Enterprise B | B-1 | Hippocampus mohnikei, Hippocampus |
| | | | camelopardalis |
| 2 | Enterprise B | B-2 | Hippocampus mohnikei, Hippocampus |
| | | | camelopardalis |
| 3 | Enterprise B | B-3 | Hippocampus camelopardalis |
| 4 | Enterprise A | A-1 | Hippocampus trimaculatus, Hippocampus |
| | | | comes, Hippocampus spinosissimus, |
| | | | Hippocampus camelopardalis |
| 5 | Enterprise A | A-2 | Hippocampus trimaculatus, Hippocampus |
| | | | comes, Hippocampus spinosissimus, |
| | | | Hippocampus camelopardalis |
| 6 | Enterprise A | A-3 | Hippocampus trimaculatus, Hippocampus |
| | | | camelopardalis |
| 7 | Enterprise A | A-4 | Hippocampus trimaculatus, Hippocampus |
| | | | camelopardalis |
| 8 | Enterprise A | A-5 | Hippocampus trimaculatus, Hippocampus |
| | | | comes, Hippocampus spinosissimus, |
| | | | Hippocampus camelopardalis |

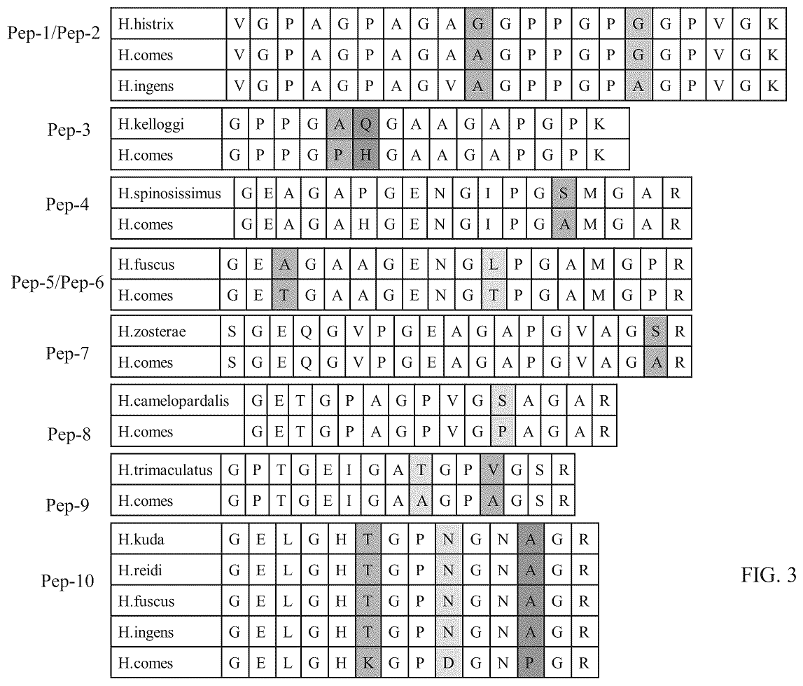

A protein content in Hippocampus was 60-70%, and collagen as a major component of connective tissue is one of the most abundant proteins. These 10 peptide biomarkers were all collagen peptides and belonged to collagen alpha-1(I) chain isoform X1 and collagen alpha-2(I) chain isoform X1. Therefore, the sequences of the corresponding site were aligned with those of the corresponding site of Hippocampus comes as shown in FIG. 3. It can be seen that individual amino acid sites of collagen are mutated during the evolution of Hippocampus species. These mutation sites can provide information for species identification. Polypeptide fragments are good carriers for carrying and displaying these different sites. The characteristic polypeptide fragments provided by the present invention may well identify the Hippocampus species involved in an unknown Hippocampus sample.

What is claimed is:

1. A method for identifying Hippocampus species using a peptide biomarker, comprising:
(1) Sample preparation
weighing a Hippocampus extract accurately, adding water, sonicating for dissolving, cooling to room temperature, measuring a supernatant accurately, and adding trypsin to obtain a test solution after enzymolysis overnight; and
(2) using High Performance Liquid Chromatography (HPLC)-triple quadrupole mass spectrometry for analysis;
wherein in step (2), parameters of the HPLC are as follows: chromatographic column Agilent Eclipse C18 with a size of 2.1×100 mm, 1.8 μm, a mobile phase A of 0.1% formic acid aqueous solution and a mobile phase B of 0.1% formic acid acetonitrile solution for gradient elution; injection of 5 μl samples at a flow rate of 0.3 ml/min;
the gradient elution is specifically as follows: 0-20 min, 3%-20% B; 20-21 min, 20%-90% B; 21-24 min, 90%-3% B; 24-30 min, 3% B;
parameters of the triple quadrupole mass spectrometry are as follows: a mode being set as a mass detector, with electrospray ionization (ESI) and positive ion multiple reaction monitoring; a flow rate of sheath gas being 46 L/h, a flow rate of auxiliary gas being 850 L/h, a spray voltage being 3.5 kV, a source temperature being 150° C., a temperature of auxiliary gas being 400° C., a cone voltage being 30 V, a collision voltage being 35 V, and a solvent delaying 0-1 min and 21-30 min for use;
a sequence of the peptide biomarker being as follows:

| Peptide | |
|---|---|
| fragment | Sequence |
| Pep1 | VGPAGPAGAGGPP(+15.99)GPGGPVGK |
| Pep2 | GPP(+15.99)GAQGAAGAP(+15.99)GPK |
| Pep3 | GPTGEIGATGPVGSR |
| Pep4 | SGEQGVP(+15.99)GEAGAP(+15.99)GVAGSR |
| Pep5 | GELGHTGPDGNAGR |
| Pep6 | GEAGAP(+15.99)GEDGIP(+15.99)GSMGAR |
| Pep7 | GETGAAGENGTP(+15.99)GAMGPR |
| Pep8 | VGPAGPAGVAGPP(+15.99)GPAGPVGK |
| Pep9 | GEAGAAGENGLP(+15.99)GAMGPR |
| Pep10 | GETGPAGPVGSAGAR |

identification principles are as follows:
(1) when only Pep1 is detected in a sample, the sample is identified as Hippocampus histrix;
(2) when only Pep2 is detected in the sample, the sample is identified as Hippocampus kelloggi;
(3) when only Pep3 is detected in the sample, the sample is identified as Hippocampus trimaculatus;
(4) when only Pep4 is detected in the sample, the sample is identified as Hippocampus mohnikei;
(5) when only Pep5 is detected in the sample, the sample is identified as Hippocampus kuda and Hippocampus reidi;
(6) when only Pep6 is detected in the sample, the sample is identified as Hippocampus spinosissimus;
(7) when only Pep7 is detected in the sample, the sample is identified as Hippocampus comes;
(8) when only Pep8 is detected in the sample, the sample is identified as Hippocampus ingens;
(9) when only Pep9 is detected in the sample, the sample is identified as Hippocampus fuscus; and
(10) when only Pep10 is detected in the sample, the sample is identified as Hippocampus camelopardalis.

2. The method according to claim 1, wherein in step (1), a specific preparation process of the Hippocampus extract is as follows: taking an appropriate amount of test Hippocampus and placing in a triangular flask, adding water to soak for 48 h, and changing the water once during the 48 h; adding 250 ml of water to desalted Hippocampus and decocting at a high temperature 3 times for 4 h, 3 h, and 2 h, respectively; combining decocted solutions, concentrating decocted solutions by slight boiling until they are viscous, transferring to a silica gel bowl, and drying to solid in a 60° C. electrothermal constant temperature air blast drying oven.


SEQUENCE LISTING (10 sequences)

SEQ ID NO: 1 - protein, 21 aa, synthetic construct
VGPAGPAGAGGPPGPGGPVGK

SEQ ID NO: 2 - protein, 15 aa, synthetic construct
GPPGAQGAAGAPGPK

SEQ ID NO: 3 - protein, 15 aa, synthetic construct
GPTGEIGATGPVGSR

SEQ ID NO: 4 - protein, 19 aa, synthetic construct
SGEQGVPGEAGAPGVAGSR

SEQ ID NO: 5 - protein, 14 aa, synthetic construct
GELGHTGPDGNAGR

SEQ ID NO: 6 - protein, 18 aa, synthetic construct
GEAGAPGEDGIPGSMGAR

SEQ ID NO: 7 - protein, 18 aa, synthetic construct
GETGAAGENGTPGAMGPR

SEQ ID NO: 8 - protein, 21 aa, synthetic construct
VGPAGPAGVAGPPGPAGPVGK

SEQ ID NO: 9 - protein, 18 aa, synthetic construct
GEAGAAGENGLPGAMGPR

SEQ ID NO: 10 - protein, 15 aa, synthetic construct
GETGPAGPVGSAGAR